(19) United States
(12) Patent Application Publication
(10) Pub. No.: US 2008/0269837 A1
(43) Pub. Date: Oct. 30, 2008

(54) IMPLANTABLE MEDICAL LEAD FOR IMPLANTATION IN A PATIENT, SUCH AS A PATIENT'S NECK

(76) Inventors: Christy Ludlow, Bethesda, MD (US); Pamela Reed Kearney, Burke, VA (US); Thomas E. Cross, St. Francis, MN (US); Robyn L. Jagler, Eagan, MN (US); Robert L. Olson, Vadnais Heights, MN (US); James Britton Hissong, Jacksonville, FL (US)

Correspondence Address: DICKE, BILLIG & CZAJA, PLLC;ATTN: MD MATTERS, FIFTH STREET TOWERS, SUITE 2250, 100 SOUTH FIFTH STREET, MINNEAPOLIS, MN, 55402, US

(21) Appl. No.: 11/742,442

(22) Filed: Apr. 30, 2007

(51) Int. Cl. A61N 1/372

(52) U.S. Cl. 607/48

(57) ABSTRACT

Implantable leads implantable in a patient, such as patient's neck. The lead includes a first lead segment and a second lead segment. The second lead segment extends from the first lead segment at a first junction point and includes first, second, and third legs each defining a longitudinal length in extension from the first junction point to a distal end. A length of the first leg is greater than a length of the second leg, and a length of the second leg is greater than a length of the third leg. With this configuration, each of the legs are adapted to support at least one electrode at the distal end thereof, and are appropriately dimensioned relative to one another for locating the corresponding electrode at a desired target tissue site in the patient's neck. Targeted tissue can include, for example, muscles of the neck.

### BACKGROUND

The present invention relates to implantable medical leads for connection between a stimulating control device and one or more stimulation or sensing electrodes, and more particularly to implantable medical leads for use in particular bodily regions, such as a patient's neck, that are adapted (e.g., sized) to interface with desired target tissue.

Systems and methods for electrical stimulation of electrically excitable tissue within the body of a living subject have been developed utilizing stimulating electrodes and a signal generator or control device to supply electrical charges in a controlled or predetermined manner. Such systems and methods have been developed specifically based upon a desired condition, such as to alleviate pain or to stimulate muscle movement, and based upon the application with a subject's body. For bodily applications where the alleviation of pain is the goal, one or more stimulating and/or sensing electrodes can be implanted within nerve tissue, the brain or spinal cord for blocking pain sensation by electrical stimulation. For muscle tissue stimulation, a stimulating electrode can be implanted in a muscle tissue, whereby electrical current that is typically provided as pulses can cause muscle tissue reaction that may be controlled to cause movement of a subject's body part. Sensing electrodes are used for determining actions of the body.

Signal generators can determine when, how long, and/or the amperage of current pulses that are to be applied for the specific application, and often include hard-wired circuitry, a microprocessor with software and/or embedded logic as the controlling system for determining and dictating current pulses. Such signal generators may also be implanted within the subject's body, and typically such an implantation is done to position the signal generator close to the stimulating and/or sensing electrodes, with interconnecting medical leads for conducting current pulses to and from the stimulating and sensing electrodes. Implantable medical leads and externally utilized leads for these purposes are typically insulated conductors or conductive elements (e.g., a conductor disposed within a lead body), with conductive terminations at both ends for electrical connection with the signal generator and one or more electrodes.

Signal generation and muscle tissue stimulation systems have more recently been envisioned for more complex control of a subject's bodily actions. To accomplish more complex movements, it has been considered to control a pattern of energization of multiple electrodes to stimulate action of distinctly different muscles in combination and/or in series. The attempt of such systems is to stimulate muscle tissue in the order of movement that reflects normal body movements that may have, for example, been lost or disabled by trauma or disease, the purpose of which may be to re-teach a subject of a particular movement or to supplement or replace the subject's control of such movement.

A particularly complex muscular control concept has recently been considered for the purpose of re-teaching a subject how to swallow, the condition of inability to swallow being known as dysphagia, which condition is a common complication with diseases such as stroke, neurodegenerative diseases, brain tumors, respiratory disorders, and the like. Dysphagia is of great concern in that the risk of aspiration pneumonia, which inflects a 20% death rate in the first year after a stroke and 10-15% each year thereafter, is very high. Prior treatments for dysphagia require either temporary feeding through a nasogastric tube or enteric feeding through a stoma to the stomach in chronic cases.

Techniques and methods of stimulating muscles within the neck region of a patient for the purposes of causing specifically determined muscles to react as a swallowing effect are described in PCT Publication No. WO 2004/028433, having a publication date of Apr. 8, 2004. Specifically, by implanting electrodes in two or more muscles of the upper airway musculature and connecting the electrodes with a signal generator that provides coordinate control signals, a swallowing action can be induced in the patient. One goal of such technique is to re-teach the patient how to swallow without such stimulation subsequent to such treatment. Other specific techniques and methods are also disclosed in U.S. Pat. Nos. 5,725,564; 5,891,185; 5,987,359; 6,104,958; and 6,198,970; all to Freed et al. Other techniques and methods are disclosed in U.S. patent application Ser. No. 11/611,367, filed Dec. 15, 2006, and entitled “Method and Apparatus for Assisting Deglutition.” The teachings of each of these references are incorporated herein by reference in their entireties.

Commensurate with the above, as well as in connection with other implanted electrode-based treatments that may or may not be related to dysphagia, implanting electrodes within disparate tissue of a patient's neck is desirable. The use of multiple electrodes on each side of the neck region of a human subject requires the running (e.g., tunneling) of multiple leads along the neck and all the way to the upper region of each side of the patient's neck from the patient's chest. Due to the relatively confined nature presented by the neck's anatomy, conventional implantable lead and lead assembly configurations may be insufficient. More particularly, one conventional approach in which multiple leads are individually extended from the implanted signal generator may be difficult to implant within the patient's neck and may lead to patient discomfort during head, neck, or swallowing movements or otherwise.

In light of the above, a need exists for a lead configuration appropriate for specific bodily regions, such as within the neck, as well as related implantable systems and methods of implanting such leads.

### SUMMARY

Some aspects in accordance with the present disclosure relate to an implantable lead that is implantable, for example, in a patient's neck. The lead includes a first lead segment and a second lead segment. The first lead segment extends from a proximal side to a distal side. The second lead segment extends from the first lead segment at a first junction point spaced from the proximal end. Further, the second lead segment includes first, second, and third legs each defining a longitudinal length in extension from the first junction point to a respective distal end. In this regard, a length of the first leg is greater than a length of the second leg, and a length of the second leg is greater than a length of the third leg. With this configuration, each of the legs are adapted to support at least one electrode at the distal end thereof, and are appropriately dimensioned relative to one another for locating the corresponding electrode at a desired target tissue site in the patient's neck. In some non-limiting embodiments, the first leg has a length of approximately 1.75 inches, the second leg has a length of approximately 1.5 inches, and the third leg has a length of approximately 1.25 inches. In other embodiments, each of the legs includes a conductive element disposed within a lead body, with the conductive elements extending from the corresponding lead body and into a lead body provided with the first lead segment. In yet other embodiments, at least a portion of the first lead segment defines a non-linear shape (e.g., sigmoid shape) along a region adjacent the first junction point to provide the lead with increased flexibility and extensibility.

Yet other aspects in accordance with the present disclosure relate to an implantable system for electrically stimulating tissue in a patient, such as in a patient's neck. The system includes a signal generator, a lead, and a plurality of electrodes. The lead is provided as above, with the first lead segment establishing an electrical connection between the first-third legs and the signal generator. Further, respective ones of the electrodes are electrically coupled to respective ones of the distal ends of the first-third legs. With this configuration, the system is amenable for implantation relative to a neck region of a patient, with at least a portion of the lead being implantable within the patient's neck. In this regard, the length relationships established by and between the first, second and third legs promote implantation of the electrodes at disparate target tissue sites of the neck, for example the geniohyoid muscle, the hyoglossus muscle, and the mylohyoid muscle. In some embodiments, the lead further includes a third lead segment extending from the first lead segment at a second junction point spaced from the first junction point, with the third lead segment including a fourth leg extending from the second junction point to a distal end adapted to support or form an electrode. With this alternative construction, the fourth leg is amenable to positioning the corresponding stimulating electrode at a desired target tissue site of the patient's neck, such as the thyrohyoid muscle.

Yet other aspects in accordance with principles of the present disclosure relate to a method of implanting a lead within a patient. The method includes providing a lead as described above, including a first lead segment and a second lead segment extending from the first lead segment at a junction point. The second lead segment includes first, second, and third legs each defining a longitudinal length in extension from the first junction point to a distal end at which an electrode is formed or provided. In this regard, a length of the first leg is greater than a length of the second leg that in turn is greater than a length of the third leg. The first, second and third electrodes are then implanted at first, second and third target tissue sites, respectively, of the patient. For example, in some embodiments, the first target tissue site is a geniohyoid muscle, the second tissue target site is a hyoglossus muscle, and the third tissue target site is a mylohyoid muscle.

### BRIEF DESCRIPTION OF THE DRAWINGS

FIG. 1 is a side view of an implantable medical lead in accordance with aspects of the present disclosure;

FIG. 2 is a cross-sectional view of a leg portion of the lead of FIG. 1;

FIG. 3 is a cross-sectional view of a first lead segment of the lead of FIG. 1;

FIG. 4 is a cross-sectional view of a junction element useful with the lead of FIG. 1;

FIG. 5 is a cross-sectional view of a second junction element useful with the lead of FIG. 1;

FIG. 6 is a perspective view of an implantable lead assembly including an implantable lead in accordance with aspects of the present disclosure; and

FIG. 7 is a simplified illustration of a patient to which a system in accordance with aspects of the present disclosure is implanted.

### DETAILED DESCRIPTION

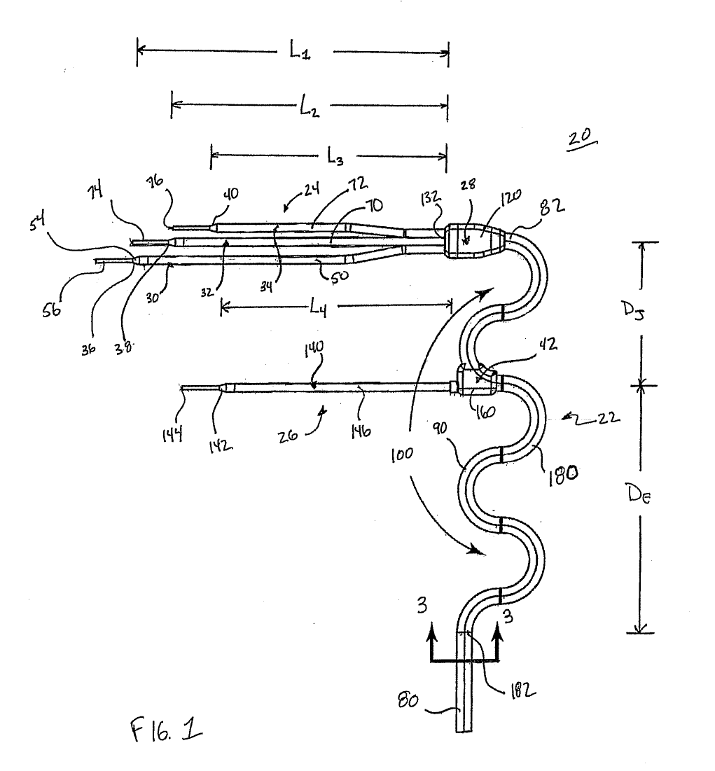

One embodiment of a lead 20 in accordance with aspects of the present disclosure is shown in FIG. 1. The lead 20 can assume a variety of forms, and includes or defines a first lead segment 22, a second lead segment 24, and an optional third lead segment 26. Details on the various components are provided below. In general terms, however, the second lead segment 24 extends from the first lead segment 22 at a first junction point 28. The second lead segment 24 includes or defines first, second, and third legs 30, 32 and 34, each having a longitudinal length in extension from the first junction point 28 to a distal end 36, 38 and 40, respectively. In this regard, the length of the first leg 30 is greater than the length of the second leg 32, and the length of the second leg 32 is greater than the length of the third leg 34. As described below, this relationship of and between the leg lengths has surprisingly been found to be beneficial for implantation in certain bodily regions, especially a patient's neck. Finally, where provided, the third lead segment 26 extends from the first lead segment 22 at a second junction point 42 that is otherwise longitudinally spaced from the first junction point 28 in promoting implantation of an electrode at a discrete target tissue site apart from electrodes associated with the first, second and third legs 30, 32 and 34.

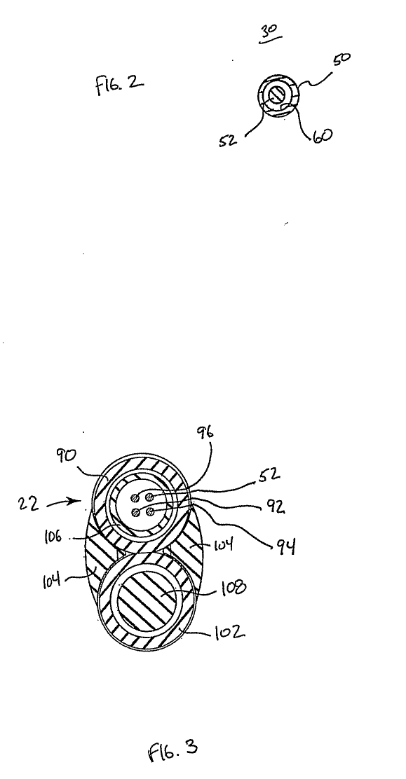

Each of the first, second, and third legs 30-34 can assume a variety of forms, and in some embodiments are substantially identical except in terms of length as mentioned above. For example, and with additional reference to FIG. 2 otherwise illustrating in cross-section one embodiment of the first leg 30 (it being understood that the second and third legs 32, 34 may be identically constructed), the first leg 30 includes a lead body 50 and at least one conductive element or conductor 52. As generally referenced in FIG. 1, the conductive element 52 forms or defines a distal conductive lead termination 54 and a proximal conductive lead termination (not shown). The distal termination 54 forms or is otherwise electrically coupleable to one or more electrodes 56 at the distal end 36, for example a stimulation electrode as known in the art. Regardless, the lead body 50 encompasses or otherwise maintains at least a distal region of the conductive element 52, such that the lead body 50 supports the distal lead termination 54. As described in greater detail below, other region or regions of the conductive element 52 (e.g., an intermediate region and/or a proximal region) may extend proximally from the lead body 50, or may be more fully encompassed by the lead body 50. However, at least the distal region of the conductive element 52 is substantially covered by the lead body 50 for containing and preferably electrically insulating the corresponding region(s) of the conductive element 52.

The lead body 50 can comprise any number of layers, which layers may be located directly on the conductive element 52 or spaced from the conductive element 52, and may include any number of functional layers. Further, while the lead body 50 is illustrated in FIG. 2 as forming or defining a lumen 60 within which the conductive element 52 is disposed, in other embodiments, the lead body 50 need not form or define a lumen within which the conductive element 52 is otherwise disposed. For example, the lead body 50 can encompass the conductive element 52 within a material thickness of the lead body 50 (e.g., the lead body 50 can be molded to the conductive element 52 that otherwise is provided in coil form, etc.). Thus, the lead body 50 can be formed from a variety of acceptable materials including, for example, silicone rubber, or any other application compatible material(s).

The conductive element 52 can comprise any known or develop conductive wire or the like that may be a solid element (e.g., shaft, coil, etc.), and/or be comprised as a stranded conductor as such are well-known. Stranded wire as used for the conductive element 52 would typically be more flexible as compared with solid wire. However, a solid wire is typically more capable of being deformed to hold a shape and can exhibit a spring-back characteristic that may be useful with leads in accordance with some embodiments of the present disclosure. The lead terminations 54 can comprise any known or developed electrical connection that may be appropriate for connection between other electronic components depending on the specific applications. For example, the distal (and/or proximal) lead termination 54 may be merely uninsulated wire portions for connection with other electrical connectors, or may comprise the connectors themselves as fixed to the end(s) of the conductive element 54. While FIG. 2 reflects the single conductive element 52 within the first lead body 50, in other embodiments, two or more of the conductive elements 52 can be provided with the lead body 50, and can be insulated from one another in a conventional manner (e.g., by insulation material coating).

With specific reference to FIG. 1, the second and/or third legs 32, 34 can have a similar or dissimilar construction to that described above with respect to the first leg 30. That is to say, one or both of the second and third legs 32 and/or 34 can assume the configuration reflected in FIG. 2, or can have an entirely different construction. In general terms, however, the second and third legs 32, 34 each include a lead body 70, 72, respectively, within which at least one conductive element (hidden in FIG. 1) is provided. The conductive element of the second leg 32 is electrically coupled to or forms at least one electrode 74 (e.g., a stimulation electrode), whereas the conductive element associated with the third leg 34 is electrically coupled to or forms at least one electrode 76.

While the legs 30-34 are illustrated in FIG. 1 as being substantially linear, in other embodiments, one or more of the legs 30, 32, and/or 34 can have a non-linear shape as described below. Regardless, the legs 30-34 are effectively coupled to one another at the first junction point 28, but are movable relative to one another in extension from the first junction point 28. That is to say, the second lead segment 24 is configured such that the distal end 36 of the first leg 30 (and thus the electrode 56 associated therewith) is movable relative to the distal ends 38, 40 of the second and third legs 32, 34; the distal end 38 of the second leg 32 (and thus the electrode 74 associated therewith) is movable relative to the distal ends 36, 40 of the first and third legs 30, 34; etc.

As mentioned above, the second lead segment 24 is constructed to provide a particular relationship between the lengths of the first-third legs 30-34 relative to one another whereby the first leg 30 is longer than the second leg 32, and the second leg 32 is longer than the third leg 34. More particularly, the first leg 30 has or defines a first length L₁ in extension from the first junction point 28 to the distal end 36. The second leg 32 similarly has or establishes a second length L₂ in extension from the first junction point 28 to the distal end 38. Finally, the third leg 34 has or defines a third length L₃ in extension from the first junction point 28 to the distal end 40. As a point of reference, the lengths L₁-L₃ are relative to the legs 30-34 being arranged or oriented in the substantially linear fashion depicted in FIG. 1. With these designations in mind, the first length L₁ is greater than the second length L₂, and the second length L₂ is greater than the third length L₃. In some embodiments, the difference in length between the first leg length L₁ and the second leg length L₂ is substantially identical to the difference in length between the second leg length L₂ and the third leg length L₃ (e.g., within 5%) although in other embodiments the difference in lengths can be varied. In yet other embodiments, the difference in the first leg length L₁ and the second leg length L₂, and/or the difference in length between the second leg length L₂ and the third leg length L₃ is in the range of 0.1-0.5 inch; alternatively, not less than 0.2 inch; alternatively not greater than 0.5 inch. In yet other embodiments, the difference in the first leg length L₁ and the second leg length L₂, and/or the difference in length between the second leg length L₂ and the third leg length L₃ is approximately 0.25 inch (+/−0.02 inch).

As described in greater detail below, in some embodiments, the second lead segment 24 provides the first leg 30 for positioning the corresponding stimulation electrode 56 in a geniohyoid muscle. For these and other applications, it has surprisingly been found that forming the first leg length L₁ to be on the order of 1.5-2.0 inches provides beneficial results in terms of least ease of implant, efficacy and/or patient comfort. In other embodiments, the first leg length L₁ is approximately 1.75 inches (+/−5%). Alternatively, other dimensions for the first leg length L₁, either greater or lesser, are also acceptable.

In some embodiments, the second lead segment 24 provides the second leg 32 for positioning the corresponding stimulation electrode 74 within a hyoglossus muscle of a patient, although other applications are also acceptable. Relative to the hyoglossus application, it has surprisingly been found that providing the second leg length L₂ to be in the range of 1.25-1.75 inches provides beneficial results. In other embodiments, the second leg length L₂ is approximately 1.5 inches (+/−5%). Alternatively, the second leg length L₂ can be greater or lesser.

In some embodiments, the second lead segment 24 is configured to provide the third leg 34 for positioning the corresponding stimulation electrode 76 within the mylohyoid muscle, although other applications are also acceptable. With this one embodiment, however, it has surprisingly been found that by forming the third leg length L₃ to be in the range of 1.0-1.5 inches provides beneficial results. In yet other embodiments, the third leg length L₃ is approximately 1.25 inches (+/−5%). Alternatively, however, the third leg length L₃ can be greater or lesser.

Commensurate with the above explanations, in some embodiments in which the lead 20 is implanted within the neck region of a patient, it has surprisingly been found that forming the first leg length L₁ to be approximately 1.75 inches (e.g., plus or minus 5%), the second leg length L₂ to be approximately 1.5 inches (e.g., plus or minus 5%), and the third leg length L₃ to be approximately 1.25 inches (e.g., plus or minus 5%) greatly promotes appropriate implantation and minimizes patient's discomfort or other complications. Alternatively, however, a wide variety of other dimensions for one or more of the leg lengths L₁-L₃ are also envisioned. Further, one or more legs in addition to the first-third legs 30-34 can be included with the second lead segment 24.

As mentioned above, the second lead segment 24 extends from the first lead segment 22. In this regard, the first lead segment 22 defines a proximal side 80 and a distal side 82. The proximal side 80 is configured to establish an electrical connection of the first-third legs 30-34 (and in particular the conductive elements provided therewith) with a separate component (e.g., implantable stimulation energy or signal generator, a lead extension connected to an implantable stimulation generator, etc.). With the one embodiment of FIG. 1, the second lead segment 24 as described above extends from the distal side 82 of the first lead segment 22. In other embodiments, the second lead segment 24 can extend from the first lead segment 22 at a point between the proximal and distal sides 80, 82. Preferably, however, the second lead segment 24 is spaced from the proximal side 80 of the first lead segment 22.

The first lead segment 22 can assume a variety of forms, and in some embodiments includes at least one lead body 90. With additional reference to FIG. 3, the lead body 90 can assume any of the forms described above, and is adapted to maintain one or more conductive elements. In some embodiments, the first lead segment 22 is configured to maintain a region of each of the conductive elements associated with the legs 30-34 (FIG. 1), and are otherwise reflected in FIG. 3 as the conductive elements 52, 92, and 94 (it being understood that the conductive element 52 is the conductive element is associated with the first leg 30, the second conductive element 92 is associated with the second leg 32, and the third conductive element 94 is associated with the third leg 34). With this construction, then, the conductive elements 52, 92, and 94 of the second lead segment 24 extend through and are carried by the lead body 90 of the first lead segment 22 for a proximal connection to a corresponding component (e.g., implantable signal generator or control device). As a point of reference, FIG. 3 further illustrates a fourth conductive element 96. In some embodiments, and as described in greater detail below, the fourth conductive element 96 extends at least partially along the lead body 90 of the first lead segment 22 and extends as part of the third lead segment 26. In other embodiments, the first lead segment 22 can include or more or less of the conductive elements 52, 92, 94 and 96 as otherwise shown.

As best shown in FIG. 1, in some embodiments, a portion of the first lead segment 22 has a non-linear shape in longitudinal extension. As described in greater detail in U.S. application Ser. No. 11/413,437 filed Apr. 28, 2006, the teachings of which are incorporated herein by reference. The non-linear shape can be defined as a shaped pattern 100 and renders the first lead segment 22 to be flexible and extensible at least along the shaped pattern 100 (or portions thereof). The pattern 100 can assume a variety of shapes, and in some embodiments is or defines a sigmoid shape. As reflected in FIG. 1, the non-linear shape is provided at or adjacent the first junction point 28 from which the second lead segment 24 otherwise extends. The shaped pattern 100 is elastically deformable under load and will return to the no-load shape (reflected in FIG. 1) once the load is removed. The purpose of allowing the shaped pattern 100 (or portions thereof) to deform elastically is to preferably provide for controlled extensibility to be designed into the lead 20, for example at the first lead segment 22, under any expected load for conditions that may be present under any specific application. For implantation of the lead 20 along a neck region of a patient, such as for treatment of dysphagia, the non-linearly shaped pattern 100 exhibits an extensibility of about 40 percent when subjected to a load force of 0.1 pounds or less, preferably less. Other applications can have very different requirements with higher or lower extensibility levels under higher or lower load values.

The two-dimensional, flexible and extensible shape pattern 100 can be imparted to the first lead segment 22 via one or more shaping elements and in a variety of fashions, numerous ones of which are described in U.S. application Ser. No. 11/413,437. In one embodiment, and as shown in FIG. 3, the first lead segment 22 includes a second lead body 102 connected with the first lead body 90 (e.g., via adhesive 104). In addition, a first shaping element 106 is provided with the first lead body 90 and a second shaping element 108 is provided with the second lead body 102. In this regard, the shaping element 106, 108 can assume a variety of forms; with the one embodiment of FIG. 3, the first shaping element 106 is a tube formed about the conductive elements 52, 92, 94 and 96, collectively disposed within the first lead body 90. The second shaping element 108 is an elongate member disposed within the second lead body 102. The shaping elements 106, 108 combine to define the shaped pattern 100 in the no-load state. In other embodiments, the first and/or second shaping elements 106 and/or 108 can assume other forms (e.g., a shaping sheet material), and one or both can be eliminated. In even further embodiments, the first lead segment 22 need not provide the non-linear shape reflected in FIG. 1, and instead can be more akin to a conventional, linear design.

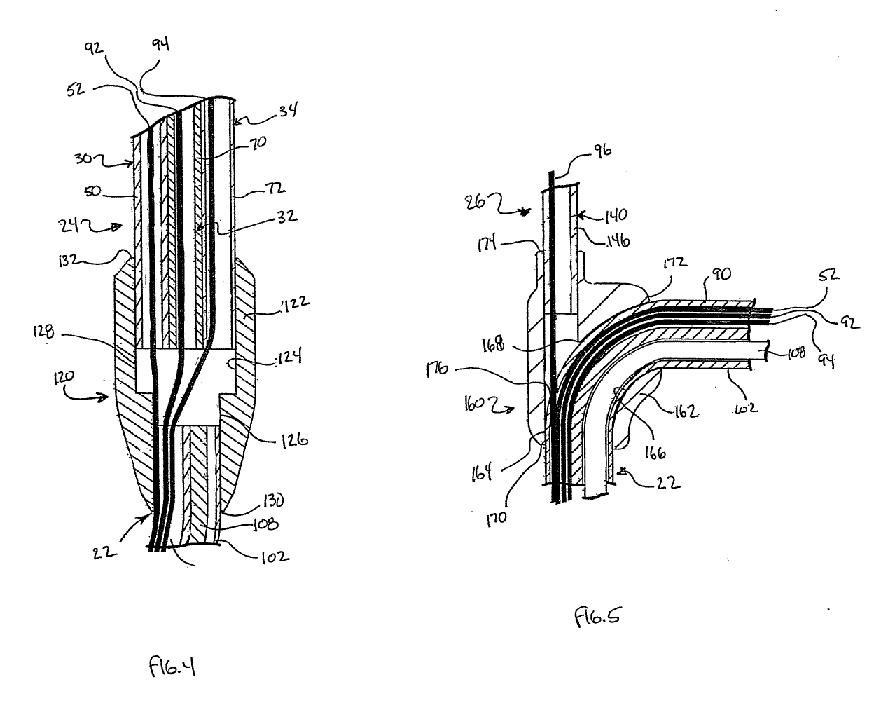

Regardless of the exact construction of the first lead segment 22, the first junction point 28 can facilitate extension of one or more conductive elements from the second lead segment 24 to the first lead segment 22. With this in mind, the first junction point 28 can be formed or defined by a junction element 120, one embodiment of which is shown in greater detail in FIG. 4. The junction element 120 includes a housing 122 forming a passage 124 having a first section 126 and a second section 128. The passage 124 extends between, and is open relative to, opposing end faces 130, 132 of the housing 122. The first section 126 is sized to receive the first lead segment 22, whereas the second section 128 is sized to receive the second lead segment 24. The housing 122 is preferably affixed (e.g., bonded) to the lead segments 22, 24. The legs 30-34 extend within, and are coupled relative to, the second section 128. The corresponding conductive elements 52, 92, 94 extend between the corresponding lead body 50, 70, 72 and the passage 124. Further, the conductive elements 52, 92, 94 extend into or from the first lead body 90 of the first lead segment 22. With this construction, then, the junction element 122 facilitates separation of the conductive elements 52, 92, 94 from the first lead segment 22 to the second lead segment 24. Alternatively, a wide variety of other components can be employed to effectuate a transition between the first and second lead segments 22, 24.

In other embodiments, a discrete junction element need not be provided in forming the first junction point 28. Where a junction element is provided, however, the above-described length characteristics associated with the legs 30-34 are, in some embodiments, relative to the point at which the legs 30-34 extend from the junction element. For example, relative to the one embodiment junction element 120, the length L₁ of the first leg 30 is defined as the dimension or distance between the end face 132 of the junction element 120 and the distal end 36. The second and third lengths L₂ and L₃ also relate to a dimension or distance from the end face 132.

With specific reference to FIG. 1, with embodiments including the optional third lead segment 26, the second junction point 42 is spaced from the first junction point 28 such that the third lead segment 26 extends from the first lead segment 22 at a point displaced from extension of the second lead segment 24. With this in mind, in some embodiments the third lead segment 26 includes a fourth leg 140 having or defining a longitudinal length L₄ in extension from the second junction point 42 to a distal end 142. As with the legs 30-34, the fourth leg 140 supports or carries an electrode 144 at the distal end 142, and can include a lead body 146 maintaining one or more conductive elements, such as the fourth conductive element 96 previously described with reference to FIG. 3. In general terms, the fourth leg 140 can assume any of the forms previously described with respect to one or more of the first-third legs 30-34, and can have the relatively linear shape illustrated in FIG. 1, or can assume a non-linear shape (e.g., akin to the non-linear shape described with respect to one optional embodiment of the first lead segment 22, and in particular the shaped pattern 100).

For embodiments in which the fourth leg 140 provides a continuation of the fourth conductive element 96 from the first lead segment 22 (or any other conductive element(s) associated at least in part with the first lead segment 22), a junction element 160 can be provided and defines, at least in part, the second junction point 42. For example, FIG. 5 illustrates one embodiment of the junction element 160 as including a case 162 defining or forming a first passage 164, a second passage 166, and a third passage 168. The first passage 164 extends from, and is open relative to, a first face 170 of the case 162. Similarly, the second passage 166 extends from, and is open relative to, a second face 172 of the case 162. Finally, the third passage 168 extends from, and is open relative to, a third face 174 of the case 162. With this construction, the passages 164-168 are commonly open to one another within the case 162.

The junction element 160 allows passage the first lead segment 22 to pass there through (e.g., via the first and second passages 164, 166), and also allows a conductive element (e.g., the fourth conductive element 96) from the first lead segment 22 to be directed into the (and form part of) the fourth leg 140 (e.g., via the third passage 168). For example, the junction element 160 can be constructed such that the lead bodies 90, 102 of the first lead segment 22 extend through the first and second passages 164, 166, with the first lead body 90 forming an opening 176 through which the fourth conductive element 96 exits the lead body 90 and is directed toward the third passage 168. In this regard, the second passage 166 is sized to receive a portion of the fourth leg 140, for example the lead body 146. As shown, then, the fourth conductive element 96 extends along the third passage 168 and within the lead body 146. Any bonding, adhesive, or other fit technique can be used for assembling the lead segments 22, 26 to the junction element 160, with the lead body or bodies associated with the first lead segment 22 (e.g., the lead bodies 90, 102) extending to and beyond the junction element 160.

Returning to FIG. 1, the junction element 160 can assume a wide variety of other forms, and in other embodiments, a discrete junction element need not be provided in forming the second junction point 42. Regardless, the second junction point 42 is longitudinally spaced from the first junction point 28 (for example, proximally spaced) by a junction separation distance D_{J}. With embodiments in which the lead 20 is provided for implantation in a patient's neck, for treatment of, for example, dysphagia, it has surprisingly been found that optimal results (in terms of ease of implant, mitigation of seromas, and/or patient comfort) can be accomplished where the junction separation distance D_{J} is in the range of approximately 0.9-1.0 inch (plus or minus 5%), for example approximately 0.95 inch (plus or minus 5%) for implantation of the electrode 144 at the thyrohyoid muscle (where others of the electrodes 56, 74, 76 are implanted at one or more other muscles of the patient's neck). As a point of reference, with embodiments in which the first lead segment 22 includes the non-linear shaped pattern 100 between the first and second junction points 28, 42, the junction separation distance D_{J} represents a linear distance between the junction points 28, 42 (as compared to a continuous length of the first lead segment 22 between the junction points 28, 42). Other values for the junction separation distance D_{J}, either greater or lesser, are also acceptable for other end-use applications.

With embodiments in which the first lead segment 22 defines the non-linear shaped pattern 100, an intermediate non-linear pattern portion 180 can be designated as extending proximally from the second junction point 42 to a leading side 182. Where provided, the pattern portion 180 defines a lateral extension distance D_{E} from the leading side 182 to the second junction point 42 having a dimension on the range of 1.2-2.0 inches, and in some embodiments approximately 1.60 inches (plus or minus 5%). It has surprisingly been found that for applications in which the lead 20 is implanted in the neck of a patient with the fourth leg 140 located to position the corresponding electrode 144 at the thyrohyoid muscle, the extension distance D_{E} of approximately 1.60 inches (plus or minus 5%) provides optimal results in terms of one or more of ease of implantation, long term placement, and patient comfort. Alternatively, the extension distance D_{E} can be greater or lesser than that described above, and in other embodiments is not present in that the first lead segment 22 does not include or form the intermediate non-linear shaped pattern portion 180.

In addition to the distances D_{J} and D_{E}, in some embodiments, defining the fourth leg length L₄ to be less than the first-third lengths L₁-L₃ has surprisingly provided beneficial results in the context of neck implantation applications, and in particular for the treatment of dysphagia. In some embodiments, the fourth leg length L₄ is in the range of 0.8-1.2 inches; and in other embodiments is approximately 1.0 inches (plus or minus 5%). Alternatively, other dimensions, either greater or lesser (including greater than one or more of the first, second and third lengths L₁, L₂ and L₃) are also acceptable.

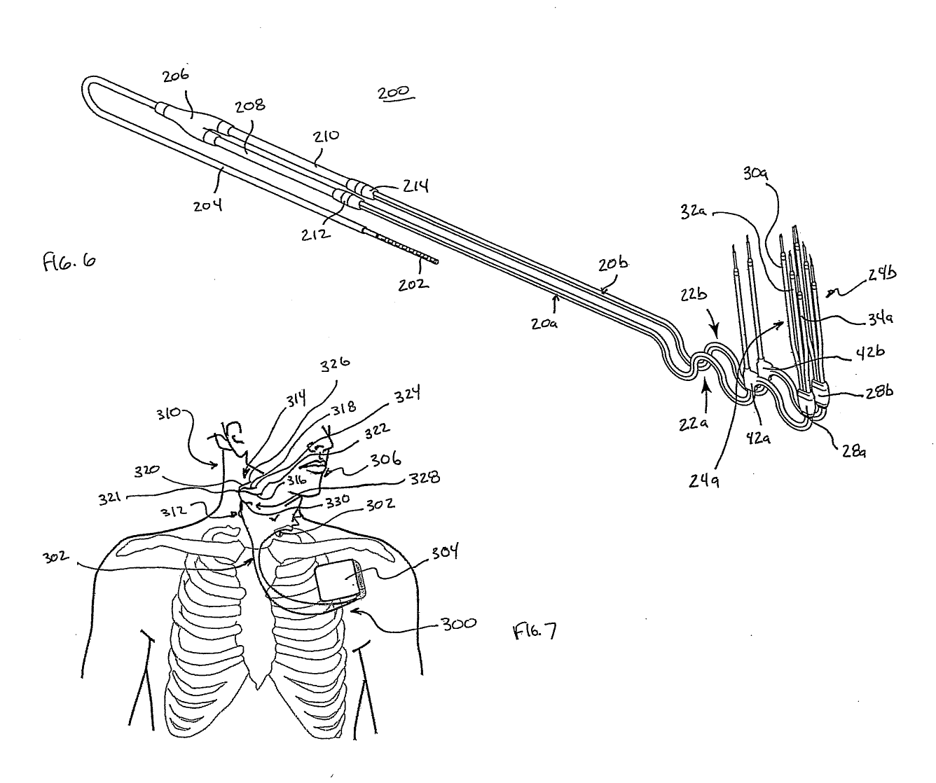

FIG. 6 illustrates one embodiment of a lead assembly 200 in accordance with aspects of the present disclosure, incorporating first and second leads 20a, 20b, each of which are, in some embodiments, akin to the lead 20 (FIG. 1) described above, with like components identified by corresponding elements numbers and the suffix “a” or “b”. At proximal end 202, an electrical termination is provided, such as may be in the form of any multiple electrical connector or jack for electrical connection of any number of conductive elements to a control unit of a signal generator (not shown). Extending distally, a first tubing 204 provides a passage for any number of insulated conductive elements that are to be used in the lead assembly 200. A splitting element 206 separates and guides one or more conductive elements into second and third tubings 208 and 210. Any number of tubings can be used for a particular application and an appropriate splitter(s) provided. At the end of the second and third tubings 208, 210, connectors 212 and 214 facilitate connection to the leads 20a, 20b that otherwise incorporate features in accordance with aspects of the present disclosure. More particularly, the first lead 20a provides a second lead segment 24a extending from a first lead segment 22a, with the second lead segment 24a including three legs 30a-34a exhibiting the length relationships described above. The second lead 20b can be similarly constructed, including three legs exhibiting the length relationships described above.

During use, and with reference to FIG. 7, an implantable stimulation system 300 can be provided, including one of more leads 302 in accordance with principles of the present invention along with an implantable signal generator 304 of a type known in the art. The system 300 can be implanted at various regions of a patient 306, and in some embodiments is implanted and employed to effectuate electrical stimulation treatments in a neck region 310 of the patient 306. Regardless, the lead(s) 302 are generally akin to those described with respect to the lead 20 of FIG. 1, and include a first lead segment 312 and a second lead segment 314 extending therefrom, with the second lead segment 314 including at least three legs 316, 318, and 320 extending from a first junction point 321 to a respective distal end 322, 324, 326 at which at least one electrode is maintained or formed. A relationship of the lengths of each of the legs 316-320, including dimensional values, corresponds with that described above. Further, the first lead segment 314 establishes an electrical coupling between the legs 316-320 (and thus the electrodes associated therewith) and the signal generator 304. In some embodiments, the lead(s) 302 may further include an optional third lead segment 328 in the form of a fourth leg 330 maintaining an electrode in electrical communication with the signal generator 304 (via the first lead segment 312, for example) and/or the non-linear shaped (e.g., sigmoid) portion of the first lead segment 312.

One application for which the system 300 is useful is in the treatment of dysphagia by providing for the electrical connection of the signal generator 304 with multiple electrodes as located or implanted for teaching the patient 306 to swallow. Implantation surgery to facilitate implantation of the system 300, and in particular the lead(s) 302 can include insertion of the lead(s) 302 through any one or more incisions that may be formed in connection with the surgery and running (e.g., tunneling) of the lead(s) 302 through or along tissue. With respect to some embodiments in which the lead(s) 302 are implanted in the neck region 310, the electrode associated with the distal end 322 of the first leg 316 is implanted into, or placed in stimulating contact with, the geniohyoid muscle; the electrode associated with the distal end 324 of the second leg 318 is implanted into, or placed in stimulating contact with, the hyoglossus muscle; and the electrode associated with the distal end 326 of the third leg 320 is implanted into, or placed in stimulating contact with, the mylohyoid muscle. In addition, the electrode associated with a distal end 332 of the fourth leg 330 is implanted into, or placed in stimulating contact with, the thyrohyoid muscle. Alternatively, a wide variety of other target tissue sites can be accessed by one or more of the electrodes associated with the system 300. With this but one application, however, the length relationships for the first-third legs 316-318 render affecting the above muscle/electrode placements straightforward; further, where the specific leg lengths described above are employed, the risk of seromas and surgical difficulties has surprisingly been found to be mitigated. Further, location of one or more of the junction points as described above allows the junction point(s) to readily be “fit” to a location of the hyolaryngeal area in the patient 306.

Although the present invention has been described with reference to preferred embodiments, workers skilled in the art will recognize that changes can be made in form and detail without departing from the spirit and scope of the present invention.

What is claimed is:

1. An implantable lead implantable in a patient, such as a patient's neck, the lead comprising:
a first lead segment extending from a proximal side to a distal side;
a second lead segment extending from the first lead segment at a first junction point spaced from the proximal side, the second lead segment comprising:
first, second, and third legs each defining a longitudinal length in extension from the first junction point to a respective distal end,
wherein the length of the first leg is greater than the length of the second leg, and the length of the second leg is greater than the length of the third leg; and
a conductive element provided with at least one of the lead segments.

2. The lead of claim 1, wherein a difference in the lengths of the first and second legs is approximately equal to a difference in the lengths of the second and third legs.

3. The lead of claim 1, wherein the length of the first leg is at least 0.2 inch greater than the length of the second leg, and the length of the second leg is at least 0.2 inch greater than the length of the third leg.

4. The lead of claim 1, wherein the length of the first leg is in the range of approximately 1.5-2.0 inches.

5. The lead of claim 4, wherein the length of the second leg is in the range of approximately 1.25-1.75 inches.

6. The lead of claim 5, wherein the length of the third leg is in the range of approximately 1.0-1.5 inches.

7. The lead of claim 6, wherein the length of the first leg is approximately 1.75 inches, the length of the second leg is approximately 1.5 inches, and the length of the third leg is approximately 1.25 inches.

8. The lead of claim 1, wherein:
the first leg includes a first conductive element disposed within a first lead body;
the second leg includes a second conductive element disposed within a second lead body; and
the third leg includes a third conductive element disposed within a third lead body.

9. The lead of claim 8, wherein the second lead segment is configured such that the distal end of the first leg is movable relative to the distal ends of the second and third legs, respectively.

10. The lead of claim 8, wherein the first lead segment establishes an electrical connection between each of the first, second, and third conductive elements and the proximal side of the first lead segment.

11. The lead of claim 10, wherein the first lead segment includes a fourth lead body, and further wherein the first, second, and third conductive elements extend within the fourth lead body.

12. The lead of claim 11, wherein the first conductive element defines a proximal termination point and a distal termination point, and further wherein the distal termination point is supported by the first lead body and the proximal termination point is supported by the fourth lead body.

13. The lead of claim 1, wherein the first junction point is formed at the distal side of the first lead segment.

14. The lead of claim 1, wherein at least a section of the first lead segment adjacent the first junction point defines a sigmoid pattern.

15. The lead of claim 1, further comprising:
a third lead segment extending from the first lead segment at a second junction point spaced from the first junction point, the third lead segment comprising a fourth leg defining a longitudinal length in extension from the second junction point to a lead electrode end.

16. The lead of claim 15, wherein the length of the fourth leg is less than a length of the third leg.

17. The lead of claim 16, wherein the length of the first leg is approximately 1.75 inches, the length of the second leg is approximately 1.5 inches, the length of the third leg is approximately 1.25 inches, and the length of the fourth leg is approximately 1.0 inch.

18. The lead of claim 15, wherein the first junction point is distal the second junction point.

19. The lead of claim 18, wherein a linear distance between the first and second junction points is in the range of approximately 0.9-1.0 inch.

20. The lead of claim 19, wherein the first lead segment defines a non-linear shape in extension between the first and second junction points in a relaxed state, and further wherein the linear distance is relative to the first lead assembly segment in the relaxed state.

21. The lead assembly of claim 15, wherein:
the first leg includes a first conductive element within a first lead body;
the second leg includes a second conductive element disposed within a second lead body;
the third leg includes a third conductive element disposed within a third lead body;
the fourth leg includes a fourth conductive element disposed within a fourth lead body; and
the first lead segment includes a fifth lead body;
wherein a portion of each of the first-fourth conductive elements extend within the fifth lead body.

22. An implantable system for electrically stimulating tissue such as in a patient's neck, the system comprising:
a signal generator;
a lead comprising:
a first lead segment extending from a proximal side to a distal side,
a second lead segment extending from the first lead segment at a first junction point spaced from the proximal side, the second lead segment comprising:
first, second, and third legs each defining a longitudinal length in extension from the first junction point to a respective distal end,
wherein the length of the first leg is greater than the length of the second leg, and the length of the second leg is greater than the length of the third leg,
wherein the first lead segment establishes an electrical connection between the first, second and third legs and the signal generator; and
a plurality of stimulating electrodes, respective ones of which are electrically coupled to respective ones of the distal ends of the first, second and third legs.

23. The system of claim 22, wherein a length of the first leg is approximately 1.75 inches, the length of the second leg is approximately 1.5 inches, and the length of the third leg is approximately 1.25 inches.

24. The system of claim 22, wherein the system is configured such that upon implantation, the first leg positions an electrode at a geniohyoid muscle of the patient, the second leg positions an electrode at a hyoglossus muscle of the patient, and the third leg positions an electrode at a mylohyoid muscle of the patient.

25. The system of claim 22, wherein the lead further comprises:
a third lead segment extending from the first lead segment at a second junction point spaced from the first junction point, the third lead segment comprising a fourth leg defining a longitudinal length in extension from the second junction point to a distal end.

26. The system of claim 25, wherein the system is configured such that upon implantation, the first leg positions an electrode at a geniohyoid muscle of the patient, the second leg positions an electrode at a hyoglossus muscle of the patient, the third leg positions an electrode at a mylohyoid muscle of the patient, and the fourth leg positions an electrode at a thyrohyoid muscle of the patient.

27. The system of claim 25, wherein:
the first leg includes a first conductive element within a first lead body;
the second leg includes a second conductive element disposed within a second lead body;
the third leg includes a third conductive element disposed within a third lead body;
the fourth leg includes a fourth conductive element disposed within a fourth lead body; and
the first lead segment includes a fifth lead body;
wherein a portion of each of the first-fourth conductive elements extend within the fifth lead body.

28. A method of implanting a lead within a patient, the method comprising:
providing a lead comprising:
a first lead segment extending from a proximal side to a distal side; and
a second lead segment extending from the first lead segment at a first junction point spaced from the proximal side, the second lead segment comprising:
first, second, and third legs each defining a longitudinal length in extension from the first junction point to a respective distal end,
wherein the length of the first leg is greater than the length of the second leg, and the length of the second leg is greater than the length of the third leg;
forming a first electrode at the distal end of the first leg;
forming a second electrode at the distal end of the second leg;
forming a third electrode at the distal end of the third leg;
implanting the first electrode at a first tissue target site of the patient;
implanting the second electrode at a second tissue target site of the patient; and
implanting the third electrode at a third tissue target site of the patient.

29. The method of claim 28, wherein the first tissue target site is a geniohyoid muscle, the second tissue target site is a hyoglossus muscle, and the third tissue target site is a mylohyoid muscle.

30. The method of claim 28, wherein the lead further includes a third lead segment extending from the first lead segment at a second junction point spaced from the first junction point, the third lead segment comprising a fourth leg defining a longitudinal length in extension from the second junction point to a distal end, the method further comprising:
forming a fourth electrode at the distal end of the fourth leg; and
implanting the fourth electrode at a fourth tissue target site of the patient.

31. The method of claim 30, wherein the first tissue target site is a geniohyoid muscle, the second tissue target site is a hyoglossus muscle, the third tissue target site is a mylohyoid muscle, and the fourth tissue target site is a thyroid hyoid muscle.
